(19) United States
(12) Patent Application Publication
(10) Pub. No.: US 2022/0304700 A1
(43) Pub. Date: Sep. 29, 2022

(54) HEMOSTASIS BAND WITH INFLATABLE COMPARTMENTS AND METHODS OF USING SAME

(71) Applicant: Terumo Medical Corporation, Somerset, NJ (US)

(72) Inventor: Victoria Moore O'Brien, Sewell, NJ (US)

(21) Appl. No.: 17/214,272

(22) Filed: Mar. 26, 2021

(51) Int. Cl. A61B 17/135

(52) U.S. Cl. CPC A61B 17/135; A61B 2017/12004

(57) ABSTRACT

The present application discloses various embodiments of a hemostasis band comprising inflatable compartments and methods of using same. These inflatable compartments provide enhanced fit and/or comfort features.

### BACKGROUND

The present invention relates to a hemostasis band adapted to act as a compression device to promote hemostasis at a surgical access site and, more particularly, to a vascular hemostasis band having one or more inflatable compartments to enhance patient comfort and/or proper fit.

After a surgical procedure involving arterial or venous access, it may be desirable or necessary to apply pressure to the access site to promote hemostasis. Existing hemostasis bands— some of which are in an annular shape—have been used in the past to apply pressure to the access site. When used on some portions of the body—for example irregular or tapered portions—such bands may tend to migrate, thus reducing the compressive effectiveness of the device. Further, in some applications such bands can dig into the patient's skin, causing discomfort.

Accordingly, there is a need for a hemostasis band that addresses these and other drawbacks of the prior art.

### SUMMARY OF THE DISCLOSURE

In one respect, the present disclosure comprises a hemostasis device comprising: a main body comprising a band, the band being adapted to be wrapped and releasably secured around a body part of a patient that includes a site where bleeding is to be stopped, the band comprising an interior side that faces a skin surface of the patient when releasably secured to the body part of the patient and at least one edge portion that comprises at least a portion of a perimeter of the band, the interior side of the band comprising at least one compartment located along the at least one edge portion, the at least one compartment being inflatable; and a compression element, the compression element comprising at least one balloon, the at least one balloon being inflatable and adapted to be placed atop the site on the body part of the patient; wherein the band acts to direct an applied force generated by the at least one balloon when it is inflated towards the site on the body part of the patient.

In another respect, the present disclosure comprises a hemostasis device comprising: a band, the band being adapted to be wrapped and releasably secured around a body part of a patient that includes a site where bleeding is to be stopped, the band comprising an interior side that faces a skin surface of the patient when releasably secured to the body part of the patient, the interior side of the band comprising a contiguous inflatable chamber, the contiguous inflatable chamber comprising at least one edge portion located along at least a portion of a perimeter of the band; and a compression element comprising at least one balloon, the at least one balloon being inflatable and adapted to be placed atop the site on the body part of the patient; wherein the at least one band acts to direct an applied force generated by the at least one balloon when it is inflated towards the site on the body part of the patient.

### BRIEF DESCRIPTION OF THE DRAWINGS

The present disclosure will hereinafter be described in conjunction with the appended drawing figures, wherein like numerals denote like elements.

FIG. 1 is a perspective view of a hemostasis band according to the prior art, in a wrapped configuration;

FIG. 2 is a front perspective view of the prior art hemostasis band of FIG. 1, shown in a fully-wrapped configuration located around a patient's wrist;

FIG. 3a is a force diagram of the prior art hemostasis band of FIG. 1, on an average wrist;

FIG. 3b is a force diagram of the prior art hemostasis band of FIG. 1, on a highly-tapered wrist;

FIG. 4 is a top plan view of a hemostasis band according to an embodiment of the present disclosure, in an unwrapped configuration;

FIG. 5 is an elevational view of a cross-section of the hemostasis band of FIG. 4, taken through line 5-5 of FIG. 4;

FIG. 6 is a front perspective view of the hemostasis band of FIG. 4, with the hemostasis band shown in a fully-wrapped configuration located around a patient's wrist;

FIG. 7 is a top plan view of a hemostasis band according to another embodiment of the present disclosure, in an unwrapped configuration;

FIG. 8 is an elevational view of a cross-section of the hemostasis band of FIG. 7, taken through line 8-8 of FIG. 7;

FIG. 9 is a top plan view of a hemostasis band according to yet another embodiment of the present disclosure, in an unwrapped configuration;

FIG. 10 is a top plan view of a hemostasis band according to still another embodiment of the present disclosure, in an unwrapped configuration;

FIG. 11 is a top plan view of a hemostasis band according to another embodiment of the present disclosure, in an unwrapped configuration;

FIG. 12 is an elevational view of a cross-section of the hemostasis band of FIG. 11, taken through line 12-12 of FIG. 11;

FIG. 13 is a front perspective view of the hemostasis band of FIG. 11, with the hemostasis band shown in a fully-wrapped configuration located around a patient's wrist;

FIG. 14 is a top plan view of a hemostasis band according to yet another embodiment of the present disclosure, in an unwrapped configuration;

FIG. 15 is a top plan view of a hemostasis band according to still another embodiment of the present disclosure, in an unwrapped configuration;

FIG. 16 is an elevational view of a cross-section of the hemostasis band of FIG. 15, taken through line 16-16 of FIG. 15;

FIG. 17 is a top plan view of a hemostasis band according to another embodiment of the present disclosure, in an unwrapped configuration;

FIG. 18A is an elevational view of a cross-section of the hemostasis band of FIG. 17, taken through line 18-18 of FIG. 17, while the band is in an unfolded configuration;

FIG. 18B is an elevational view of a cross-section of the hemostasis band of FIG. 17, taken through line 18-18 of FIG. 17, while the band is in a folded configuration; and

FIG. 19 is a top plan view of a hemostasis band according to yet another embodiment of the present disclosure, in an unwrapped configuration.

### DETAILED DESCRIPTION OF THE EMBODIMENT(S)

The ensuing detailed description provides exemplary embodiment(s) only, and is not intended to limit the scope, applicability, or configuration thereof. Rather, the ensuing detailed description of the exemplary embodiment(s) will provide those skilled in the art with an enabling description for implementing these embodiment(s). It should be understood that various changes may be made in the function and arrangement of elements of the embodiment(s) without departing from the spirit and scope of the invention, as set forth in the appended claims.

Directional terms (e.g., upper, lower, left, right, etc.) may be used herein. These directional terms are merely intended to assist in disclosing the embodiment(s) and claiming the invention and are not intended to limit the claimed invention in any way. In addition, reference numerals that are introduced in the specification in association with a drawing figure may be repeated in one or more subsequent figure(s) without additional description in the specification, in order to provide context for other features.

For purposes of the present specification and claims, the term “inflatable” should be understood to mean fillable with a fluid, including but not limited to air.

Further, for purposes of the present specification and claims, the term “access site” should be understood to refer to a site where arterial, venous, or other vascular access has occurred on a patient.

Hemostasis bands that are wrapped around a patient's limb at a site on the limb where bleeding is to be stopped are well known in the art. These hemostasis bands typically include one or more inflatable balloons or bladders that are disposed below a rigid plate to target pressure at a vascular access site. This pressure is usually achieved via hoop stress ensured by the secured hemostasis band, and helps achieve hemostasis at the access site. Multiple embodiments of one such hemostasis band and methods of using such devices are described in U.S. Pat. No. 7,498,477, which is incorporated by reference herein as if set forth in its entirety. Such devices are commonly an annular or cuff-like hemostasis band, and when deployed on a patient's limb or any other tapered surface (e.g., a foot), they may be prone to migration (i.e., movement), thus reducing the effectiveness of the device.

In order for a hemostasis device to properly function, the main or “belt” portion of the hemostasis band needs to do two things: (1) stay in its desired position with the pressure-supplying portion (e.g., a balloon assembly or other compression element) located atop the surgical access site; and (2) direct pressure toward the artery or vein to create hemostasis. Therefore, because hemostasis bands are often secured in anatomical areas that flex (e.g., a wrist or foot), a good fit is necessary to maintain hemostatic pressure as a patient moves. To direct pressure toward a target artery or vein, the hemostasis band is usually designed to resist stretching and, thus, when the one or more balloon(s) are inflated, the band applies pressure toward the vascular access site (e.g., an artery or vein). That is, the belt portion of the band does not simply stretch away from the access site as the one or more balloon(s) are inflated. Therefore, although elasticized belts or bandages may stay in place on the anatomy when flexed, and tend to be comfortable to patients, in some applications elasticized belts are unsuitable because the hemostasis band must resist stretching away from the access site enough to provide equal and opposite hemostatic force against the access site as the one or more balloon(s) are inflated, while not acting as a tourniquet on the patient's anatomy (i.e., not obstructing blood flow). Consequently, because existing hemostasis bands are made of inelastic materials, they are unyielding on a patient's skin and, thus, commonly cause discomfort to the patient.

Further, existing hemostasis devices typically include a rigid plate that is configured so that at least one compression element (e.g., one or more inflatable balloon) is located between the rigid plate and at least one artery or vein where an access site is located. Thus, hemostasis bands are generally configured to provide an equal and opposite force to the at least one compression element, resulting in adequate hemostatic pressure at the access site. However, the strap portion of these bands are commonly non-configurable (i.e., fixed or pre-defined with respect to their location, angle, and length). Therefore, in some instances, such as for example where the hemostasis device is being used on irregular or highly-tapered anatomy, the use of existing single strap hemostasis bands result in fit and performance limitations (e.g., migration of the band, inefficient application of force, and discomfort.

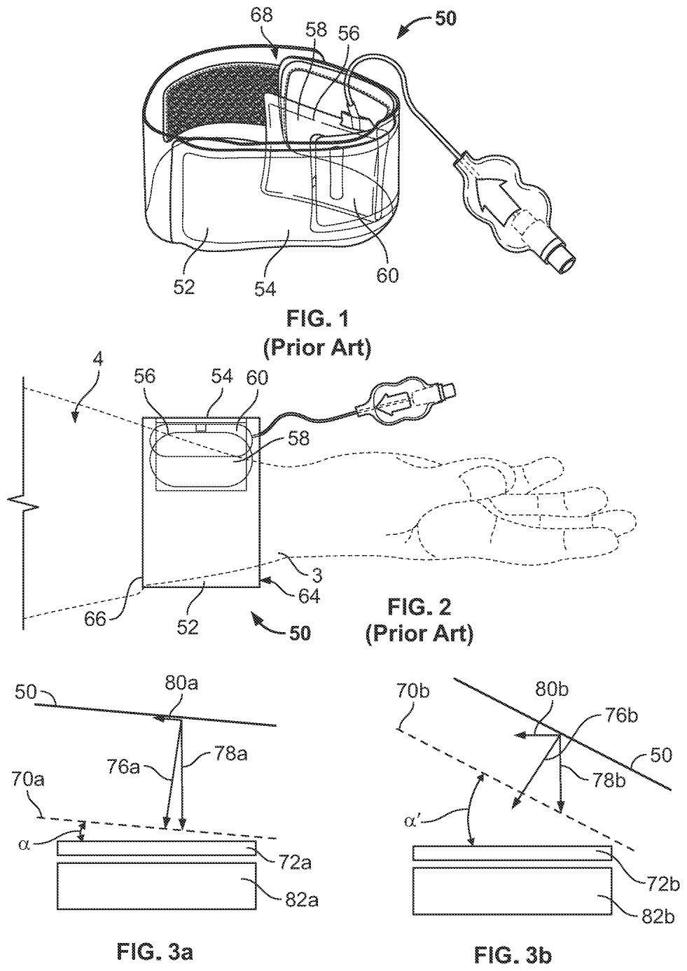

Referring to FIG. 1, a hemostasis band 50 having a single strap in accordance with the prior art is shown. The hemostasis band 50 (i.e., the TR Band sold by Terumo Kabushiki Kaisha of Tokyo, Japan), as well as some other known hemostasis bands, are indicated for use on the radial artery. Accordingly, these products are designed to fit the wrist area, while directing the force of the compression element thereof appropriately to the radial artery and while protecting the ulnar area from uncomfortable pressure. In this prior art device, the hemostasis band 50 includes a main body 52, a rigid plate 54, and a compression element 56 that includes two inflatable balloons 58,60. The hemostasis band 50 and other known hemostasis bands, however, have their drawbacks. For example, on tapered or irregular anatomy, known hemostasis bands (e.g., the TR Band) have several fit challenges.

Referring now to FIG. 2, if the hemostasis band 50 is wrapped straight around an anatomy so that it loosely (or comfortably) fits a large, tapered anatomy (e.g., an obese wrist 3), most of the surface area of the strap portion of the main body 52 does not contact or provide a force on the wrist 3, and a gap 64 may be present between the wrist 3 and the hemostasis band 50. As further described below, this gap 64 causes uneven force distribution, which may cause the band 50 to gradually slide out of position (i.e., towards the narrower part of the anatomy), which can cause bleeding (i.e., loss of hemostasis) and potential related complications. On the other hand, if the hemostasis band 50 is wrapped straight around the wrist 3, but tight enough to prevent the band 50 from sliding out of place, an edge 66 of the hemostasis band 50 may dig into the patient's skin, which may cause discomfort to the patient. A gap 64 may still be present when tightly wrapped straight around tapered anatomy (e.g., an obese wrist 3).

In an attempt to match a contour of a tapered or other irregular anatomy, such as the tapered wrist 3 or a foot, the hemostasis band 50 according to the prior art may be wrapped in a frustoconical shape around the anatomy (not shown), i.e., such that axial centerlines of the two connectable halves of the strap are not aligned, but instead oriented at an angle to each other. However, in this configuration, fastener 68 (e.g., hook and loop fastener patches; see FIG. 1) disposed on ends of the hemostasis band 50 may have reduced overlap, which results in the hemostasis band 50 being less securely attached to the patient. In some configurations, the halves of the fastener 68 (e.g., hook and loop fastener patches) might not overlap at all, thus providing no way to close the hemostasis band 50 around the patient's anatomy. Further, when a hemostasis band according to the prior art is wrapped in such an angled manner, the compression balloon(s) thereof will sit at a different angle with respect to the access site. Thus, the band may not direct the applied hemostatic force (i.e., the force generated by the inflated balloon(s)) normal to the vascular access site in the intended manner. Since this applied force (or some significant component thereof) is angled with respect to angle that is normal to the vascular access site, this angled component of the force can encourage (i.e., push) the band out of position (typically, towards the thinner portion of a tapered anatomy, for example an obese wrist 3).

Referring now to FIGS. 3a and 3b, as discussed above with regard to a single strap on tapered or irregular anatomy, the hemostasis band 50 is prone to migration (i.e., movement) even without patient movement due to the force distribution that results from a tapered anatomical surface. For example, FIG. 3a illustrates a force distribution experienced on a patient with a below average body fat percentage (e.g., below 50th percentile). The patient's skin surface 70a is slightly angled (see angle α) relative to an artery 72a. When the hemostasis band 50 is deployed on the skin surface 70a, it applies a total force 76a normal to the skin surface 70a. Only a vertical component 78a of the total force 76a is directed normal to the artery 72a and the underlying bone 82a, whereas a horizontal component 80a of the total force 76a is directed tangential to the artery 72a. In this example, because the angle α is small, the vertical component 78a is close in value to the total force 76a applied by the hemostasis band 50, with the horizontal component 80a having a relatively small value. Thus, in this configuration of the hemostasis band 50, the quantity of force that is acting to potentially move the band 50 down the tapered surface is relatively small. On the other hand, FIG. 3b illustrates a force distribution experienced on an individual with a higher body fat percentage (e.g., above 50th percentile or obese in nature). In this embodiment, skin surface 70b is shown at angle α′ relative to the artery 72b and the underlying bone 82b, with angle α′ being significantly greater than angle α shown in FIG. 3a. Therefore, the value of the force applied normal to the artery (i.e., vertical component 78b) is less than the value of the vertical component 78a shown in FIG. 3a when the total forces 76a,76b are equal. Accordingly, in this embodiment, the quantity of the force that acts to potentially move the band 50 down the tapered surface (i.e., horizontal component 80b) is relatively large, and migration of the band 50 is much more likely. In general terms, based on trigonometric relationships, as angle α′ increases, the value of the vertical component 78b of the total force 76b decreases and the value of the horizontal component 80b of the total force 76b increases.

Accordingly, based on the aforementioned drawbacks with the prior art devices and methods of using same, a need exists for a hemostasis band having increased fit capabilities, especially on tapered or irregular anatomies. In particular, a need exists for a hemostasis band with “gap-filling” and/or leveling capabilities that can be configured to enhance comfort while preventing unintentional dislocation of the device. Accordingly, embodiments of hemostasis bands according to the present disclosure include one or more inflatable compartments, which may be in the form of pillows, pockets, chambers, sections, channels, tubing, or other elements that may help to increase patient comfort and/or provide an improved fit on tapered or irregular anatomy. These inflatable compartments can help to fill gaps that may be present between a skin-facing side of a hemostasis band and the patient's skin and/or reorient (or “level”) a plane of the skin-facing side of a hemostasis band so that the compression element (e.g., one or more inflatable balloons) are oriented properly with respect to the vascular access site on the patient.

Including inflatable sections along one or more edges of a hemostasis band (i.e., along some portion of the perimeter thereof, including on the skin-facing or interior side of the band) increases comfort by creating a radiused (or otherwise enlarged) edge that distributes the force of the band at its edge over a larger area, while lifting the edge away from the skin surface. Including inflatable sections along other parts of the band (e.g., along its midline) can also increase patient comfort by contacting anatomy that would otherwise be spanned by a wrapped and closed band, via location and filling of the inflatable section(s) within the spanned gap(s). These inflatable sections distribute the force of the band over a larger area of the skin surface, thus improving patient comfort and reducing the likelihood of migration of the band. Inflatable edges or sections may also help level the one or more balloons of the compression element, to keep the applied pressure normal (in the proximal-distal direction) to an artery or other vascular access site, instead of aligned along a taper of the anatomy. Said another way, when the at least one inflatable compartment on the band is inflated, an orientation of the interior side of the band with respect to the skin surface of the patient is adjusted.

As will be described in greater detail below, various embodiments of the present disclosure can provide these and other benefits which are lacking in the prior art devices and methods. For example, embodiments of hemostasis devices according to the present disclosure can conform to various anatomies and patient sizes to attain a secure and level fit, while providing enhanced comfort to the patient, in comparison to the prior art devices. For that purpose, hemostasis bands according to some embodiments of the present disclosure may include one or more of the following components or features, either alone or in combination: one or more inflatable compartment(s)—in addition to the inflatable hemostatic balloon assembly itself—that help properly direct the pressure provided by the hemostasis band to a vascular access site (though it should be understood that, in some embodiments, the one or more inflatable compartment(s) could be connected in fluid flow communication with the hemostatic balloon assembly); one or more inflatable chamber(s) located along one or more edge(s) of the band that help with patient comfort and/or improved distribution of pressure created by the band; and/or one or more adjustable valves or clamps that provide heightened customization capabilities by allowing for the one or more inflatable chamber(s) or compartment(s) to be selectably and adjustably inflated to a desired internal pressure, to provide an appropriate tightness and angle of the band for a secure and effective fit of the band on a patient.

Various embodiments according to the present disclosure are presented below in the context of hemostasis bands intended to target pressure at a vascular access site to achieve hemostasis at that site. Although several configurations of a hemostasis band are presented herein, it should be understood that other configurations are possible within the scope and spirit of the present disclosure. For example, the principles disclosed herein can be used with other devices that use straps or bands and that require or could benefit from leveled or directed pressure over a surface, including bandages, compression sleeves, vascular closure devices for either general or specific anatomical applications, or the like. One or more removable, inflatable compartment pieces having a fastening means (e.g., a hook-and-loop fastener patch) thereon and which are locatable on various locations on a hemostasis device could also be included in an application-specific or generalized hemostasis device kit, in accordance with the scope and spirit of the present disclosure.

In some embodiments according to the present disclosure, the inflatable sections or edges can be contiguously connected to the one or more compression balloon(s), thus providing one inflation point for ease of use. In these embodiments, simultaneous inflation of the compression balloon(s) and inflatable sections or edges will occur. In alternative embodiments according to the present disclosure, the compression balloon(s) are not connected in fluid flow communication with the inflatable sections or edges, thus requiring more than one inflation point for each separately sealed component of the hemostasis device. The compression balloon(s) and the inflatable edge(s) and/or section(s) can be connected in series, in parallel, or some combination of the two, and can be physically separate (i.e., such that each component requires an independent inflation point), connected via a closeable valve, or connected via non-closeable channels.

The various inflatable edges and sections can be separately manageable via the use of valves, clamps, stopcocks, manifolds, stopcock manifolds, or other suitable means. In this way, a user can close off any inflatable portion of the band that they do not want immediately filled, inflate the portion of the band they do want filled to a desired internal size or pressure, and then close off that inflated portion before moving to the next portion. In configurations where the inflatable portions are attached in series, the user would start at the portions most distal from the inflation port, fill each portion as desired, seal it, and then work their way in a step-wise fashion towards the portions located most proximal to the inflation port. In configurations where the inflatable portion are attached in parallel (e.g., like with a manifold), the user would be able to individually open each portion, fill it as desired, and then seal it.

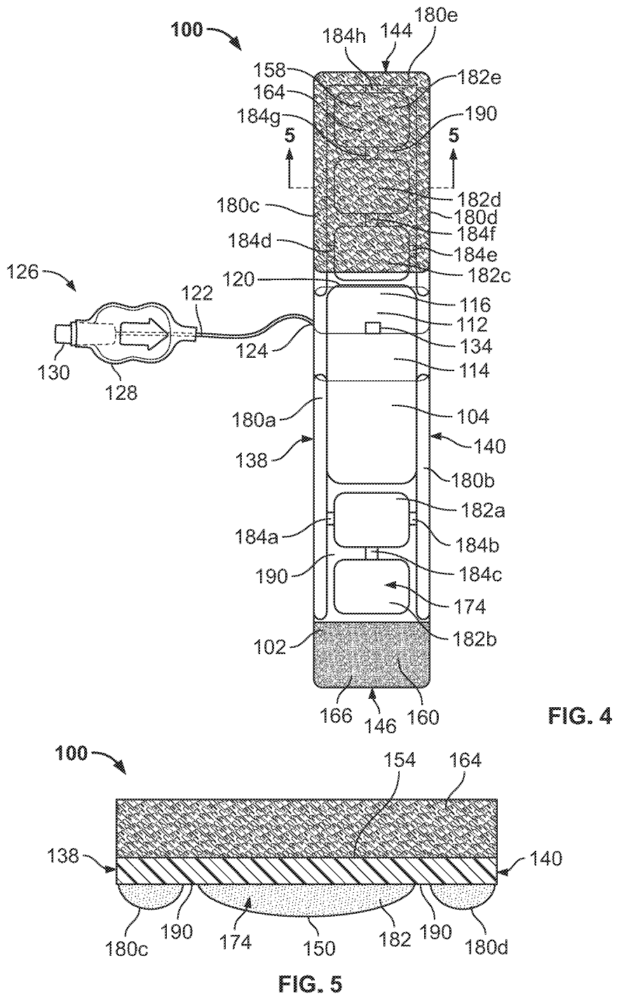

Referring now to FIGS. 4-6, an embodiment of a hemostasis band 100 according to the present disclosure will now be described in detail. In this embodiment, the hemostasis band 100 has inflatable chambers that conform to a patient's anatomy and help provide leveled and distributed pressure of a compression element (i.e., a hemostatic balloon assembly) to said anatomy. In this embodiment, hemostasis band 100 includes a main body 102 including a band (or strap) that is designed to be wrapped and secured in place around portions of an anatomy of a patient (e.g., an arm or a foot).

The hemostasis band 100 also comprises a rigid plate 104 attached to the main body 102. In this embodiment, the rigid plate 104 is held in the hemostasis band 100 between two layers of overlapping material (not separately labeled in FIG. 4) that comprise portions of the main body 102. It should be understood that, in alternative embodiments according to the present disclosure, the rigid plate 104 could be affixed to the remainder of the main body 102 by other means, for example stitching, welding, bonding, gluing, or overmolding, or could form the entirety of a portion of the main body 102 (e.g., a portion of the fastener that holds the main body 102 in place around the selected anatomy).

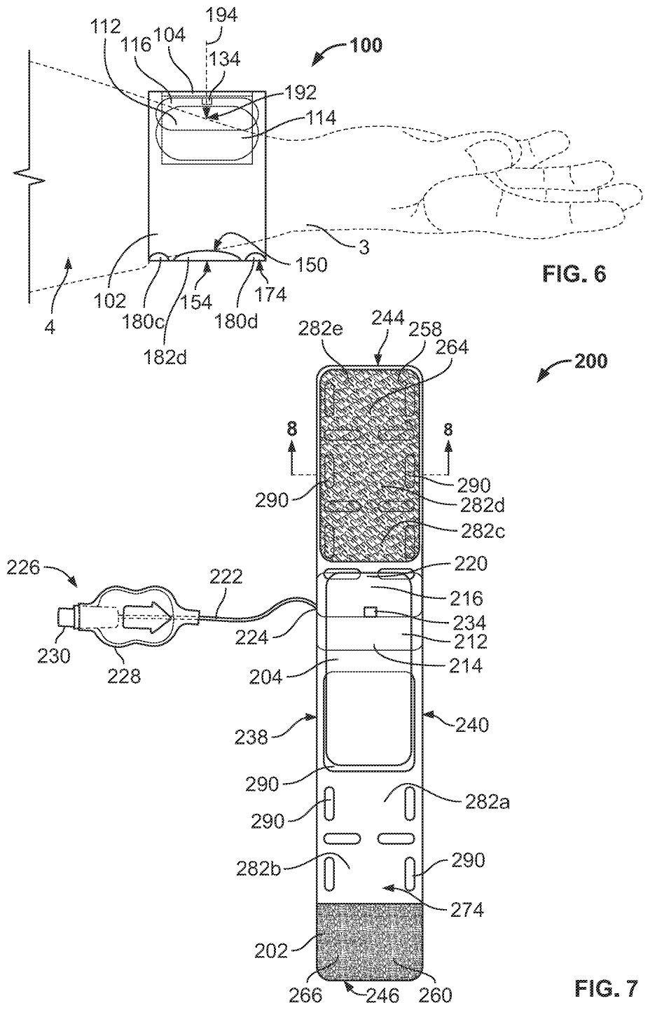

In the embodiment of FIGS. 4-6, the rigid plate 104 is made of a material that is more rigid than that of the remainder of the hemostasis band 100, and is designed to maintain a substantially constant shape. The rigid plate 104 is provided to direct pressure towards a patient's anatomy and can be used to align to specific anatomy and/or protect adjacent anatomy, as would be understood by a person having ordinary skill in the art. In this embodiment, the rigid plate 104 is configured so that at least one compression element, as further discussed below, is located between the rigid plate 104 and at least one artery or vein where an access site is located. In some embodiments, the rigid plate 104 may be a curved plate that is adapted to wrap around at least a portion of anatomy (see, e.g., wrist 3 shown in FIG. 6) when the hemostasis band 100 is attached thereto. Instead of a curved plate, a substantially planar rigid plate(s), semi-rigid non-compliant layer(s), or a combination thereof may be used in accordance with alternative embodiments of the present disclosure.

The hemostasis band 100 according to the embodiment of FIGS. 4-6 further includes a compression element 112 which is used to supply pressure to a vascular access site on a portion of a patient (e.g., on a patient's wrist 3). In this embodiment, the compression element 112 is an inflatable dual-balloon configuration similar to the embodiments taught in U.S. Pat. No. 7,498,477. This dual pneumatic balloon design supports a process to titrate air and reduce compression (i.e., pressure) during recovery, which allows for a targeted artery or vein to remain patent over time. In this embodiment, the compression element 112 comprises a main balloon 114, a secondary balloon 116, and a connector 120 which serves as a joining portion between the main body 102, the main balloon 114, and the secondary balloon 116. In the present embodiment, the connector 120 is connected about edges of each of the balloons 114,116, so that the balloons 114,116 are both hingedly attached to the main body 102 of the hemostasis band 100. The balloons 114,116 are thus integral with the main body 102 in the illustrated embodiment. However, in alternative embodiments, the connector 120 may be configured so that the balloons 114,116 are releasably attached to the main body 102 by way of fasteners, such as, for example, hook-and-loop type, snaps, buttons, hook-and-eyelet, or the like. Further, in some embodiments, the balloons 114,116 may be attached to the rigid plate 104. In further alternative embodiments, the balloons 114,116 may be connected to the hemostasis band 100 in a different fashion than what is shown, for example via their respective axial centerlines so that the balloons 114,116 inflate in a “stacked” configuration, as opposed to a “fanned” configuration as in the embodiment of FIGS. 4-6. Suitable balloon assemblies may also comprise various folded balloon designs. Various embodiments of folded balloons designs for a hemostasis device are taught in U.S. Patent Application No. 62/812,436, filed Mar. 1, 2019, the contents of which are incorporated herein by reference as if set forth in its entirety. It should be understood that the balloons 114,116 of the embodiment of FIGS. 4-6, as well as the compression elements (i.e., balloon assemblies) of the alternative embodiments taught in FIGS. 7-19, have been illustrated in a simplified fashion in the Figures for clarity, such that some features (e.g., edge seams of the balloons, connection seam(s) between the balloons and the band/rigid plate, and the internal opening between the two balloons) are not shown in the Figures. The placement and functionality of the balloon assemblies according to the present disclosure would be understood by a person having ordinary skill in the art, based on the details provided in the Figures and the remainder of the present disclosure.

In the embodiment of FIGS. 4-6, an opening (not shown) exists between an interior of the main balloon 114 and an interior of the secondary balloon 116 to allow for concurrent inflation of the main balloon 114 and secondary balloon 116, since the balloons 114,116 are connected in fluid flow communication. In some alternative embodiments (not shown), the main balloon 114 and the secondary balloon 116 may each connect separately to the main body 102 via separate connectors such that each balloon 114,116 has an individual hinge with respect to the main body 102. In some embodiments, the compression element 112 could be formed with only one balloon, any number of balloons, and/or balloon(s) of different sizes than the balloons 114,116 of the illustrated embodiment. The balloon(s) can also include one or more non-compliant material layer(s) located opposing the skin-facing side of the balloon assembly, which limits expansion of the balloon(s) outwardly from the access site. In further alternative embodiments, the balloons could be omitted entirely, and the hemostatic pressure could be achieved via a material pad or a mechanical device that applies pressure to the desired vascular access site.

Still referring to FIG. 4, a tube 122 enters an interior of the main balloon 114 via a port 124, and the tube 122 is connected at its opposite end to an inflator 126. In the present embodiment, the inflator 126 includes a bulb 128 and a valve housing 130. Inflation of the balloons 114,116 thus is achieved by inserting the protruding tip of a syringe (not shown) into the valve housing 130 and pushing a plunger on the syringe so as to introduce fluid (e.g., air) within the syringe through the inflator 126, into the balloons 114,116. Once fluid has been injected into the balloons 114,116 and the protruding tip of the syringe has been withdrawn from the valve housing 130, a check valve (not shown) within the valve housing 130 closes, preventing the fluid from leaking out and thus maintaining the balloons 114,116 in an inflated state. In the embodiment illustrated, when both balloons 114,116 are inflated, the secondary balloon 116 provides oblique pressure against the main balloon 114, which in turn provides pressure to the respective artery or vein to promote hemostasis. It should be understood that, while all of the inflators used in the embodiments described herein are substantially identical in functionality and structure and are of known type, in alternative embodiments according to the present disclosure any suitable inflation device or method could be used.

Turning back to the embodiment of FIGS. 4-6, in this embodiment the compression element 112 further includes a marker 134 located on an interior edge of the secondary balloon 116 (and approximately in the center of the main balloon 114), which permits the clinician to align the balloons 114,116 over the center of the vascular access site (arteriotomy). In alternative embodiments, the marker 134 could be placed on another surface of the hemostasis band 100, such as, for example, the center of the main balloon 114, the main body 102, or the rigid plate 104. Alternatively, the marker 134 could be located off-center so that the clinician is indicated to align the marker 134 with the visual puncture site on the patient's skin surface 4. Alternatively, the marker 134 could be omitted entirely.

Still referring to the embodiment of FIGS. 4-6, in FIG. 4 the hemostasis band 100 is shown in its undeployed or unwrapped state, lying flat on a planar surface. The hemostasis band 100 includes a perimeter that is substantially rectangular in shape and is measured along a first long edge 138, a first short edge 144, a second long edge 140, and a second short edge 146. More specifically, the first short edge 144 and the second short edge 146 are disposed between and connect to opposing ends of the first and second long edges 138,140. In this embodiment, each of the first and second short edges 144,146 and the first and second long edges 138,140 are substantially straight and have a linear length (not labeled in the figures). It should be understood, however, that the main body 102 could embody other shapes in alternative embodiments. For example, the long edges 138,140 could be curved to define a C-shaped or S-shaped body. Additionally, any of the edges 138,140,144,146 could include irregular, jagged, scalloped, or otherwise-interrupted geometries.

Further, in alternative embodiments according to the present disclosure, the hemostasis band 100 could include more edges or additional straps extending therefrom. Said another way, although all of the embodiments disclosed herein illustrate a single strap, it should be understood that multiple straps (whether separable or permanently-connected) having inflatable compartments or other elements according to the present disclosure could be provided in alternative embodiments. These straps can be pivotable and/or attached to the first strap (i.e., main body 102) at any angle, permitting additional customization for different-sized people, relative sizes of different anatomical areas, and tapered or irregularly-shaped anatomies. Correspondingly, in some embodiments, the main body 102 could be referred to as a “first strap,” and second, third, fourth, etc. straps could construct the hemostasis band 100.

As best seen in FIG. 6, the hemostasis band 100 has an interior side 150 that faces the skin surface 4 of a patient and an exterior side 154 that faces away from the skin surface 4 of the patient when the hemostasis band 100 is wrapped around the patient's wrist 3. Referring back to FIG. 4, the main body 102 is characterized by a first end 158 proximate the first short edge 144 and a second end 160 proximate the second short edge 146. In the present embodiment, each of the ends 158,160 includes a respective fastener half 164,166, which are configured to mate together to secure the hemostasis band 100 in a wrapped configuration around the selected anatomy. In the present embodiment—as well as in all of the other embodiments taught herein—the fastener halves 164,166 are patches of hook-and-loop type fasteners. In alternative embodiments according to any of the embodiments taught in the present disclosure, the fasteners could be formed from any suitable type of fastener, such as, for example, hook-and-loop fastener patches, snaps, buttons, laces, zippers, or hook-and-eyelet combinations.

The main body 102 of the hemostasis band 100 includes a contiguous inflatable chamber 174 integrally formed therein. For example, the hemostasis band 100 according to the present embodiment comprises edge portions 180a-180e, sections 182a-182e which are not located along the edges 138,140,144,146 of the main portion 102, and channels 184a-184h connected therebetween as will be discussed in further detail below, each of which is inflatable and in fluid-flow communication with the balloons 114,116. In this embodiment, the edge portions 180a,180c are formed along the long edge 138, the edge portions 180b,180d are formed along the long edge 140, and the edge portion 180e is formed along the short edge 144. In this embodiment, the edge portions 180a,180c and edge portions 180b,180d do not continue along the entirety of the respective long edges 138,140, but are instead interrupted in the areas where the balloons 114,116 are located. In alternative embodiments according to the present disclosure, the inflatable band edges may run continuously along the long edges 138,140 and/or the short edge 146, or may form any portion of the respective length thereof (for example a majority or greater than 75% of the length thereof). Although, in the present embodiment, each of the edge portions 180a-180e are clearly definable compartments, they are connected in fluid flow communication to each other by way of the various sections 182a-182e, channels 184a-184h, and the balloons 114,116 (in the areas where the edge portions 180a-180d overlap with the balloons 114,116 in FIG. 4).

In particular, in this embodiment, the contiguous inflatable chamber 174 is connected together in the following fashion: edge portions 180a,180b are each connected to the balloons 114,116 and also to section 182a via channels 184a,184b, respectively; section 182a is connected to section 182b via channel 184c; edge portions 180c,180d are each connected to the balloons 114,116 and also to section 182c via channels 184d,184e, respectively; section 182c is connected to section 182d via channel 184f; section 182d is connected to section 182e via channel 184g; and section 182e is connected to edge portion 180e via channel 184h. Because all of these elements are connected together in fluid flow communication, the contiguous inflatable chamber 174—which includes each of the edge portions 180a-180e, sections 182a-182e, and channels 184a-184h—will inflate substantially simultaneously along with the balloons 114,116. In this embodiment, the fluid that is introduced into the contiguous inflatable chamber 174 flows to the path of least resistance and will, when employed on a patient's anatomy, automatically fill gaps caused by irregular or tapered anatomy.

In all of the embodiments taught herein, the hemostasis band (which includes the one or more contiguous inflatable chambers and the compression element (e.g., one or more balloon(s)) is transparent to allow for visualization and monitoring of the vascular access site for bleeding during the hemostasis period. In alternative embodiments according to any embodiment according to the present disclosure, all or portions of the hemostasis band and/or compression element could be formed from opaque and/or semi-opaque materials. In further alternative embodiments according to any embodiment of the present disclosure, only the portions of the hemostasis band that are to be located in the vicinity of the access site(s) could be formed from transparent materials, and the remainder of the hemostasis band could be formed from opaque and/or semi-opaque materials.

Furthermore, for any of the embodiments taught herein, the hemostasis band may be made of a plasticized polymer film, such as, for example, polyvinyl chloride (PVC). For example, the hemostasis bands may be formed out of a tubular section of film. That is, a tubular section of material, such as, for example, vinyl film, could be pressed flat and welded to define the relevant inflatable edge portions, sections, channels therebetween, and any welded portions located therebetween through which a fluid is not permitted to flow. In alternative embodiments according to any embodiment of the present disclosure, other suitable materials could be used to form the hemostasis band. The hemostasis bands according to the present disclosure could also be formed through multiple methods, including for example folding a single sheet of film in half and welding it in the desired locations to form the one or more contiguous inflatable chamber(s), or welding two or more individual sheets together in a similar fashion. In further alternative embodiments according to the present disclosure, the main body of the hemostasis device may be formed of multiple pieces of material and/or a combination of different materials.

Turning back to the embodiment of FIGS. 4-6, and as perhaps best seen in FIG. 5, the edge portions 180a-180e, sections 182a-182e, and channels 184a-184h are defined by welded regions (generally indicated by reference numerals 190) disposed therebetween and through which fluids are not permitted to flow. In this embodiment, and in some other embodiments of hemostasis devices taught herein, the welded regions are narrower than the width of the inflatable edge portions. In other embodiments, the welded regions are wider than or equal in width to the inflatable edge portions.

FIG. 6 shows the hemostasis band 100 wrapped around the wrist 3 of a patient's arm, located atop an access site 192 (e.g., an access site on the radial artery). In this embodiment, the edge portions (e.g., edge portions 180c,180d) and sections (e.g., section 182d) of the contiguous inflatable chamber 174 are provided to level the hemostasis band 100 on irregular, tapered, or deformed surfaces (e.g., the obese wrist 3 shown in FIG. 6). In FIGS. 5 and 6, the contiguous inflatable chamber 174 is shown in an inflated state. When inflated, the various edge portions 180a-180e, sections 182a-182e, and channels 184a-184h conform to the patient's anatomy, thereby providing comfort and leveling of the band 100. More specifically, the inflatable band sections 182a-182e create a thicker band, which fill gaps between the hemostasis band 100 and the tapered anatomical surface (e.g., the wrist 3; compare with the unfilled gap 64 of the prior art embodiment of FIG. 2) and the inflatable edge portions 180a-180e prevent binding, digging in, or pinching of the skin surface 4 that can result from thin, uninflated edges like those found on conventional hemostasis bands. Further, as a result of the use of inflatable edge portions 180a-180e and sections 182a-182e, the hemostasis band 100 will sit more securely on the wrist 3 so that hemostatic forces are distributed more evenly to the access site 192.

While the hemostasis band 100 is secured to the wrist 3, the hemostasis band 100 can be inflated to achieve a desired fit and pressure. In the present embodiment, the balloons 114,116 and the contiguous internal chamber 174 inflate substantially simultaneously to fill the gap between the hemostasis band 100 and the tapered wrist 3. Filling this gap provides a substantially continuous contact surface between the hemostasis band 100 and the wrist 3, which distributes pressure to the wrist 3. As a result, forces that are applied by the compression element 112 to the wrist 3 (also referred to as “applied forces” herein), generally indicated by arrow 194, are redirected to be substantially normal (in the proximal-distal direction) to an artery located below the skin surface 4 at the access site 192. Further, because the forces 194 are directed normal (or significantly more normal) to the artery, and because the hemostasis band 100 contacts a greater surface area than would conventional devices, more friction exists between the hemostasis band 100 and the wrist 3, which results in a lower risk of migration of the hemostasis band 100 down the tapered surface of the wrist 3.

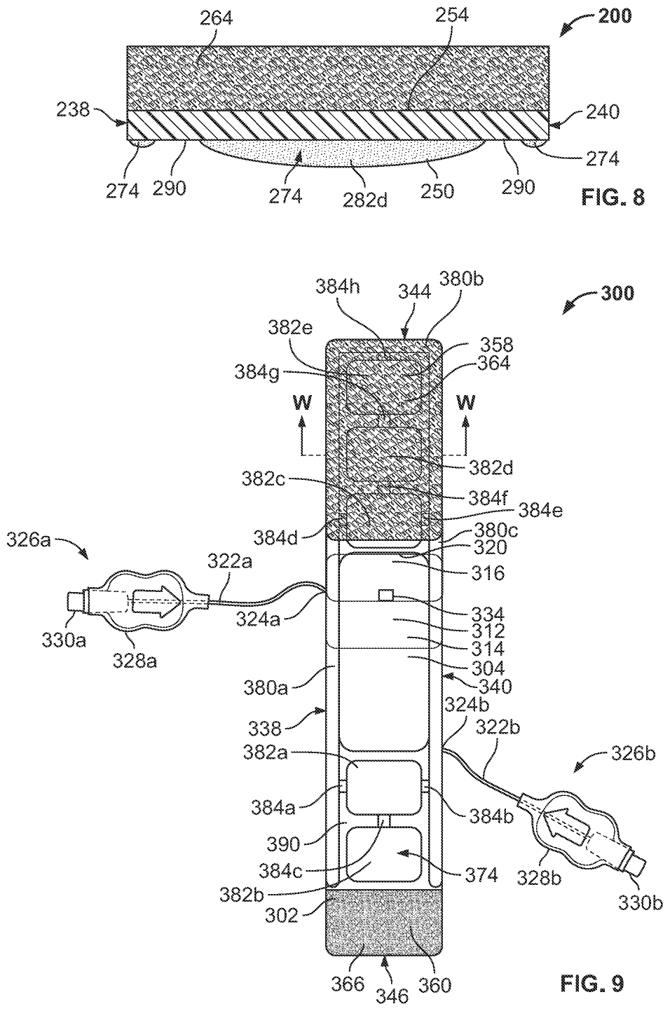

FIGS. 7-8 illustrate a hemostasis band 200 according to another embodiment of the present disclosure. Multiple elements of the hemostasis band 200 are identical or substantially-equivalent in design and functionality to those of the hemostasis band 100 shown in FIGS. 4-6. These elements are identified in FIGS. 7-8 using reference numerals increased by a value of 100 with respect to the elements of the embodiment of the hemostasis band 100 shown in FIGS. 4-6, except as described herein or as would be apparent from the figures to a person having ordinary skill in the art. Accordingly, some or all of the identical or substantially-equivalent elements of the embodiment of FIGS. 7-8 may not be discussed below, for sake of conciseness.

In this embodiment, the hemostasis band 200 includes a main body 202, a rigid plate 204, and a compression element 212. The rigid plate 204 and the compression element 212 are substantially identical to the rigid plate 104 and compression element 112 of FIG. 4. Further, the main body 202 is substantially similar to the main body 102 of FIG. 4, but it differs in some respects. For example, the main body 202 has a substantially rectangular shape and includes a contiguous inflatable chamber 274 having an overall shape that is different from that of the contiguous inflatable chamber 174 of the embodiment of FIGS. 4-6, but that is similar in cross section along significant portions of the length of the main body 202 (i.e., along an axis parallel to the long edges 238,240; see cross-sectional view of FIG. 8 taken along line 8-8 of FIG. 7).

In the hemostasis band according to the present embodiment, the contiguous inflatable chamber 274 is constructed of less clearly-defined sections or compartments that are located along either the edges or towards a center of the band, but is instead defined by a generally-inflatable cavity (i.e., the contiguous inflatable chamber 274) that extends to each of the long edges 238,240 and the first short edge 244 and across much of the center regions of the main body 202, and is interrupted only in the welded regions (generally indicated by reference numerals 290) disposed therebetween and through which fluids are not permitted to flow. In this embodiment, the welded regions 290 do generally define inflatable sections 282a-282e located towards the center of the main body 202, each having multiple spaces (not labeled) surrounding the welded regions 290 which effectively serve as channels that allow for fluid flow-communication between the inflatable sections 282a-282e the regions of the contiguous inflatable chamber 274 that extend along the long edges 238,240 and the first short edge 244. These inflatable edge portions of the hemostasis band 200 are not separately labeled in the Figures, but are indicated using reference numeral 274 in FIG. 8. The entirety of the contiguous inflatable chamber 274—including the inflatable edge portions which in this embodiment are formed along the entirety of the long edges 238,240 and the first short edge 244—is connected in fluid flow communication with the balloons 214,216 (e.g., in the vicinity of the connector 220), so that the contiguous inflatable chamber 274 and the balloons 214,216 will inflate substantially simultaneously.

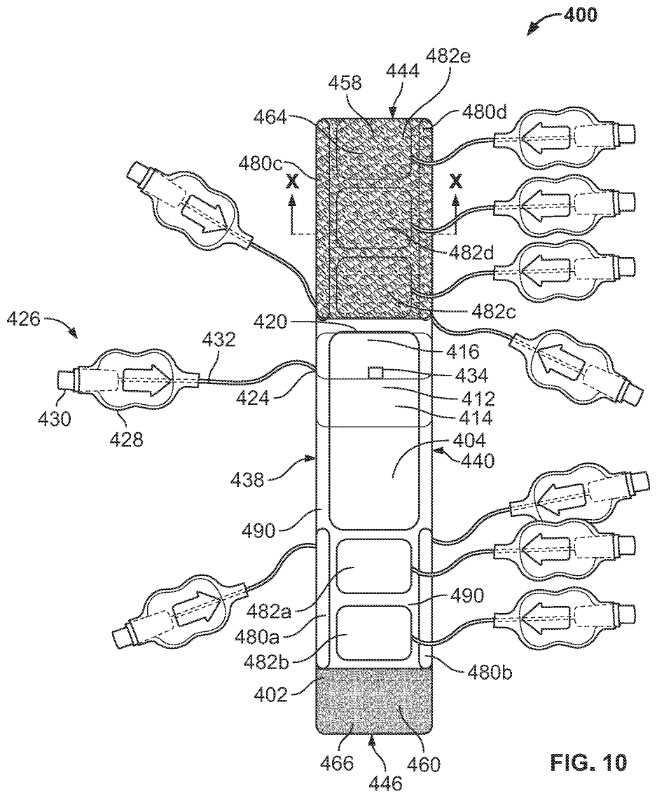

FIGS. 9 and 10 respectively illustrate hemostasis bands 300,400 according to two additional embodiments of the present disclosure. Multiple elements of the hemostasis band 300 are identical or substantially-equivalent in design and functionality to those of the hemostasis band 100 shown in FIGS. 4-6, with identical or substantially-equivalent elements identified in FIG. 9 using reference numerals increased by a value of 200 with respect to the elements of the embodiment of the hemostasis band 100 shown in FIGS. 4-6, except as described herein or as would be apparent from the figures to a person having ordinary skill in the art. Similarly, multiple elements of the hemostasis band 400 are identical or substantially-equivalent in design and functionality to those of the hemostasis band 100 shown in FIGS. 4-6, with identical or substantially-equivalent elements identified in FIG. 10 using reference numerals increased by a value of 300 with respect to the elements of the embodiment of the hemostasis band 100 shown in FIGS. 4-6, except as described herein or as would be apparent from the figures to a person having ordinary skill in the art. Accordingly, some or all of the identical or substantially-equivalent elements of the embodiments of FIGS. 9 and 10 may not be discussed below, for sake of conciseness.

In the embodiment of the hemostasis band 300 shown in FIG. 9, the main body 302 includes a contiguous inflatable chamber 374 that comprises inflatable edge portions 380a-380c (which in this embodiment represent a continuous portion connected in fluid flow communication along the long edges 338,340 and the first short edge 344, but in alternative embodiments need not), inflatable sections 382a-382e, and inflatable channels 384a-384h that are arranged substantially identically to the equivalent components illustrated in FIG. 4. Accordingly, a cross sectional view taken along line W-W of FIG. 9 is substantially like the cross-sectional view of the hemostasis band 100 taken along line 5-5 shown in FIG. 5. However, in the embodiment of FIG. 9, the contiguous inflatable chamber 374 is not connected in fluid flow communication to the balloons 314,316 of the compression element 312, such that these two elements must be inflated separately. Instead, in this embodiment, a first inflator 326a is used to inflate the balloons 314,316 of the compression element 312, and a second inflator 326b is used to inflate the contiguous inflatable chamber 374.

In the embodiment of the hemostasis band 400 shown in FIG. 10, the main body 402 does not include any contiguous inflatable chamber, but rather a collection of separate compartments which are not in fluid flow with each other or with the balloons 414,416 of the compression element 412. Instead, the compression element 412 (i.e., balloons 414,416), each of the edge portions 480a-480d, and each of the sections 482a-482e (all of which are arranged substantially identically to the equivalent components illustrated in FIG. 4, but in the absence of any connecting channels) are individually inflatable via respective individual inflators. Only one inflator, i.e., inflator 426 that is used to inflate the compression element 412, is labeled in FIG. 10 for the sake of clarity, though all of the individual inflators and their respective connecting tubes are shown in FIG. 10. A cross-sectional view taken alone line X-X of FIG. 10 is substantially like the cross-sectional view of hemostasis band 100 taken along line 5-5 shown in FIG. 5.

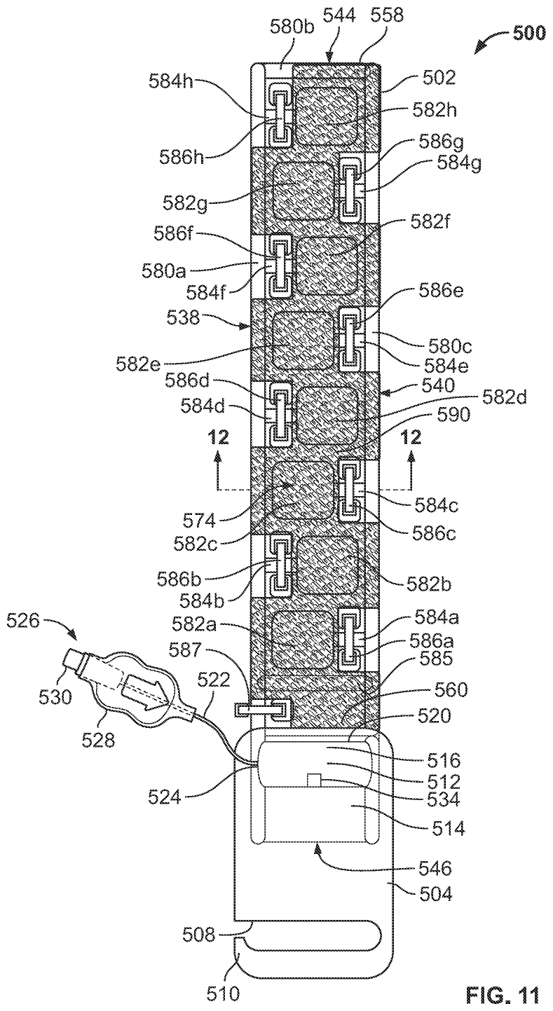

FIGS. 11-13 illustrate a hemostasis band 500 according to still another embodiment of the present disclosure. In this embodiment, elements that are identical or substantially-equivalent in design and functionality to those of the hemostasis band 100 shown in FIGS. 4-6 are shown with reference numerals increased by a value of 400 with respect to the elements of the embodiment of the hemostasis band 100 shown in FIGS. 4-6, except as described herein or as would be apparent from the figures to a person having ordinary skill in the art. Accordingly, some or all of these identical or substantially-equivalent elements may not be discussed below, for sake of conciseness.

The hemostasis band 500 includes a main body 502 that is designed to be wrapped and secured in place around portions of a patient's anatomy (e.g., an arm or a foot), as would be understood by a person having ordinary skill in the art. Similarly to the aforementioned embodiments, the hemostasis band 500 comprises a rigid plate 504 attached to the main body 502. However, in the present embodiment, the rigid plate 504 is disposed at a second end 560 of the main body 502, rather than towards the center thereof. Further, the rigid plate 504 includes a notch 508 that defines a latch 510 that is configured to attach to an opposing first end 558 of the main body 502 to secure the hemostasis band 500 in place after it has been wrapped around a portion of the patient's anatomy. The rigid plate 504 thus operates to direct pressure towards a patient's anatomy via a compression element 512 (comprising balloons 514,516), while also functioning as a fastener for closure of the hemostasis band 500. For example, in some embodiments, the latch 510 may mate with a slot or loop (not shown) disposed at the first end 558 of the main body 502. Additionally, or alternatively, the latch 510 may be configured to mate with any of one or more loops or slots disposed at different locations along a length of the main body 502, thus allowing for the hemostasis band 500 to be fastened to patients or anatomies of differing sizes. In addition, one of ordinary skill in the art would understand that the rigid plate 504 configuration illustrated in FIG. 11 could be used with hemostasis bands according to any other embodiment of the present disclosure.

In the embodiment shown in FIGS. 11-13, the main body 502 includes a contiguous inflatable chamber 574 that comprises inflatable portions 580a-580c (which in this embodiment represent a continuous portion connected in fluid flow communication along the long edges 538,540 and the first short edge 544, but in alternative embodiments need not), inflatable sections 582a-582h, and inflatable channels 584a-584h. In this embodiment, each of the inflatable sections 582b,582d,582f,582h is directly connected to inflatable edge portion 580a along the first long edge 538 via a respective one of the inflatable channels 584b,584d,584f,584h, and each of the inflatable band sections 582a,582c,582e,582g is directly connected to inflatable edge portion 580c along the second long edge 540 via a respective one of the inflatable channels 584a,584c,584e,584g, thus forming an alternating left-right pattern of connections along substantially the entire length of the main body 502. In alternative embodiments according to the present disclosure, the channel/section connections to the inflatable edge portions could be in any possible order, the section 582h could be additional or alternatively connected to the inflatable edge portion 580b, and/or the alternating pattern of inflatable sections could extend along only a portion of the length of the main body 502. In the embodiment of FIGS. 11-13, the contiguous inflatable chamber 574 is also in fluid flow communication with the balloons 514,516 of the compression element 512, so that they may inflate substantially simultaneously. The hemostasis band 500 according to the present embodiment also includes a channel 585 that connects the inflatable edge portions 580a,580c together in fluid flow communication in the vicinity of the second end 560. In the present embodiment, the inflatable edge portions 580a,580c effectively serve two roles, providing edge comfort and acting as a manifold system for the single inflator 526 to distribute air throughout the various elements of the contiguous inflatable chamber 574.

The hemostasis band 500 according to the present embodiment also includes a plurality of clamps 586a-586h,587. Clamp 587 allows for the balloons 514,516 of the compression element 512 to be selectively separated from being in fluid flow communication with the contiguous inflatable chamber 574 via sealing of the inflatable edge section 580a based on compression thereof, and each of the clamps 586a-586h allows for a respective one of the sections 582a-582h to be selectively separated from being in fluid flow communication with the remainder of the contiguous inflatable chamber 574 via sealing of a respective one of the channels 584a-584h based on compression thereof. Because the channels 584a-584h are arranged in parallel, each of the sections 582a-582h can be selectively inflated and then closed off via its respective clamp 586a-586h, thus allowing for each section 582a-582h to be sealed when a desired internal pressure has been achieved. Further, in this embodiment, the tube 522 is disposed at the compression element 512 (i.e., between the compression element 512 and the clamp 587), so that the inflator 526 can be used to adjust the hemostatic pressure in the balloons 514,516 after clamp 587 has been closed, without affecting the inflation pressure of any of the edge portions 580a-580c or sections 582a-582h. In alternative embodiments, the inflator 526 could have its tube 522 attached elsewhere to the hemostasis band 500, for example directly to the edge portion 580a with the clamp 587 located between the tube 522 and the balloons 514,516.

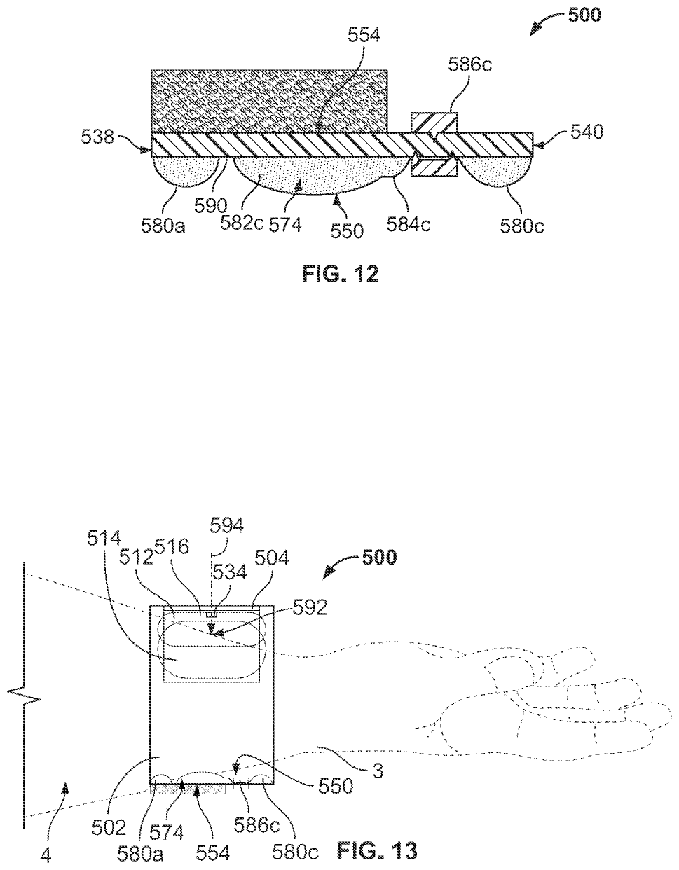

FIG. 12 shows a cross-sectional view of the hemostasis band 500 of FIG. 11, taken along line 12-12 thereof, with the contiguous inflatable chamber 574 in a fully-inflated state. FIG. 13 shows the hemostasis band 500 wrapped around a wrist 3 of a patient's arm, located atop a radial arterial access site 592, as would be understood by a person having ordinary skill in the art. In other instances, the hemostasis band 500 may be wrapped around other areas of a patient, such as, for example, a patient's foot. The hemostasis band 500 illustrated operates substantially similarly to the hemostasis band 100 illustrated in FIGS. 4-6. When wrapped around the wrist 3, the contiguous inflatable chamber 574 may be inflated to provide pressure on the wrist 3 over a large contact surface. Further, distribution of a fluid within the contiguous inflatable chamber 574 may be adjusted as desired by the clinician using the clamps 586a-586h,587.

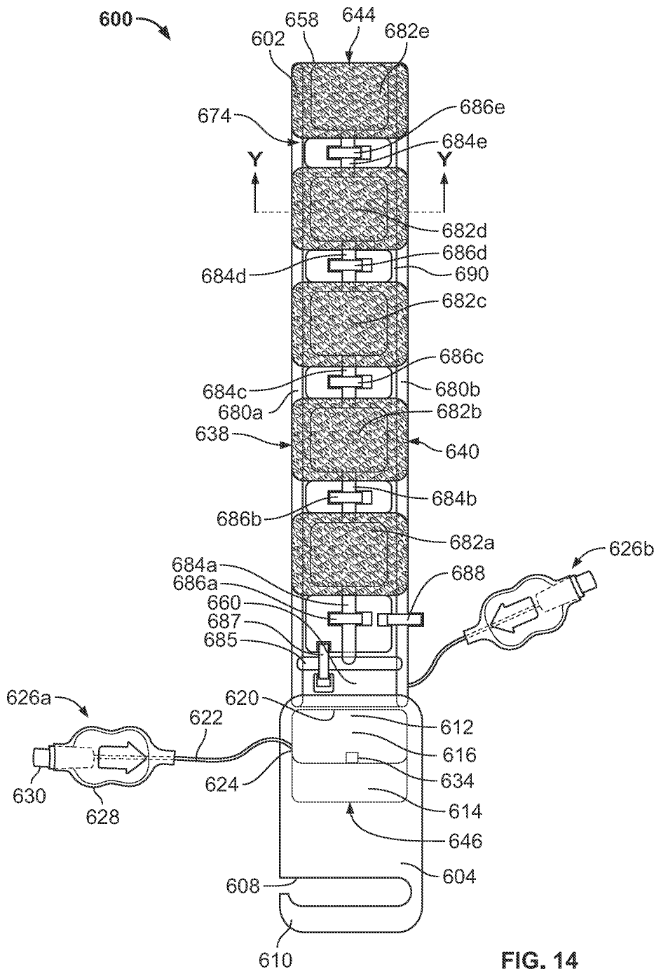

FIG. 14 illustrates a hemostasis band 600 according to still another embodiment of the present disclosure. The hemostasis band 600 is similar in design and functionality to the hemostasis band 500 shown in FIGS. 11-13, with identical or substantially-equivalent elements using reference numerals increased by a value of 100 with respect to the embodiment of FIGS. 11-13, except as described herein or as would be apparent from the figures to a person having ordinary skill in the art. Accordingly, some or all of the identical or substantially-equivalent elements of the embodiment of FIG. 14 may not be discussed below, for sake of conciseness.

The hemostasis band 600 comprises a main body 602, a rigid plate 604, and a compression element 612. Similar to the hemostasis band 500 of FIGS. 11-13, the rigid plate 604 is attached to the main body 602 at a second end 660 thereof, and the rigid plate 604 includes a notch 608 that defines a latch 610 that is configured to attach to an opposing first end 658 of the main body 602 to secure the hemostasis band 600 in place after it has been wrapped around a portion of the patient's anatomy.

In this embodiment, instead of having a contiguous inflatable chamber that is fluidly connected to the compression element—as in the hemostasis band 500 of FIGS. 11-13—the hemostasis band 600 of the current embodiment includes a contiguous inflatable chamber 674 and a compression element 612 that are not fluidly connected, such that an inflator 626a is used to inflate the compression element 612 (i.e., balloons 614,616) and a separate inflator 626b is used to inflate the contiguous inflatable chamber 674.

The main body 602 is substantially similar to the main body 102 of FIG. 4. For example, a cross-sectional view taken along line Y-Y of FIG. 14 is substantially identical to a cross-sectional view of hemostasis band 100 taken along line 5-5 of FIG. 5. However, the main body 602 differs in some respects. In this embodiment, the main body 602 includes a contiguous inflatable chamber 674 that comprises inflatable edge portions 680a,680b which are located, respectively, along the long edges 638,640, inflatable sections 682a-682e, and inflatable channels 684a-684e.

In this embodiment, a channel 685 connects the inflatable edge portions 680a,680b together in fluid flow communication in the vicinity of the second end 660, and the inflator 626b is connected to the contiguous inflatable chamber 674 between clamp elements for each of the inflatable edge portions 680a,680b. In this embodiment, a clamp 687 is located along the length of the channel 685, and the clamp 687 allows for the edge portion 680a to be selectively separated from being in fluid flow communication with the remainder of the contiguous inflatable chamber 674 via compression of the channel 685. In addition, a clamp 688 is located along the length of the edge portion 680b and allows for most of the edge portion 680b to be selectively separated from being in fluid flow communication with the remainder of the contiguous inflatable chamber 674 via compression thereof. In alternative embodiments, adjustments to the locations of either or both of the clamps 687,688 are possible, as would be appreciated by one having ordinary skill in the art.

The hemostasis band 600 according to the present embodiment also includes a plurality of sections 682a-682e which are connected to the channel 687 in series along a length of the main body 602 of the band 600 via a succession of channels 684a-684e. Each of the channels 684a-684e includes a respective clamp 686a-686e. In this embodiment, because the sections 682a-682e and channels 684a-684e and are arranged in series, the sections 682a-682e must be adjusted in series. For example, in the illustrated embodiment, as the inflator 626b is used to inflate the contiguous inflatable chamber 674, the section 682e located most distal from the inflator 626b would be adjusted and clamped first (using clamp 686e). The user would then work inwardly from there, i.e., towards section 682a, inflating and clamping off each of the remaining sections 682d,682c,682b,682a one at a time once each has reached a desired internal pressure.

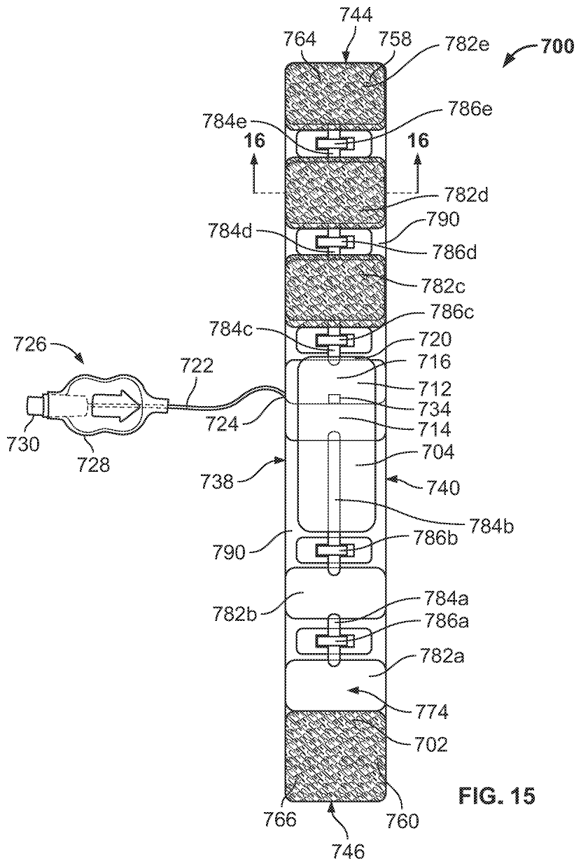

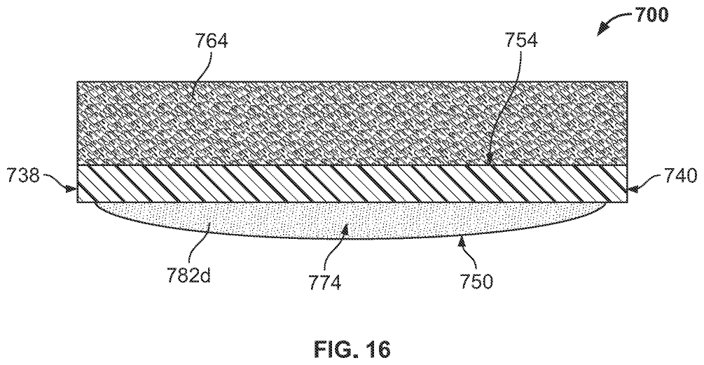

FIGS. 15 and 16 illustrate a hemostasis band 700 according to yet another embodiment of the present disclosure. The hemostasis band 700 is similar in design and functionality to the hemostasis band 100 shown in FIGS. 4-6, with identical or substantially-equivalent elements using reference numerals increased by a value of 600 with respect to the embodiment of FIGS. 4-6, except as described herein or as would be apparent from the figures to a person having ordinary skill in the art. Accordingly, some or all of the identical or substantially-equivalent elements of the embodiment of FIGS. 15 and 16 may not be discussed below, for sake of conciseness.

In the hemostasis band 700 of the present embodiment, the compression element 712 is located towards the middle of the main body 702, and is in connected in fluid flow communication, in series, to sections 782a-782e which are arranged along a length of the main body 702. In this embodiment, sections 782a and 782b are located on one side of the compression element 712 and sections 782c-782e are located on the opposite side of the compression element 712. In this embodiment, each of the inflatable sections 782a-782e is connected to each other by a plurality of inflatable channels 784a-784e that are arranged in series, thereby creating a contiguous inflatable chamber 774 that is connected with the balloons 714,716, such that all of these elements are inflatable substantially concurrently. In the illustrated embodiment, each of the inflatable sections 782a-782e extends across substantially the entirety of the width of the main body 702 (i.e., between the first long edge 738 and the second long edge 740; see section 782d in FIG. 16).

Because the inflatable channels 784a-784e are arranged in series, the sections 782e,782a proximate, respectively, the first and second ends 758,760 will typically be adjusted and isolated first via respective clamps 786e,786a. That is, a user would apply respective clamps 786e,786a to the most distal channels 784e,784a, and work inwardly from there, inflating each of the remaining sections 782d,782c,782b and clamping off each of the remaining, respective channels 784d,784c,784b via respective clamps 786d,786c,786b to seal each of the respective sections 782d,782c,782b. Moreover, since the tube 722 of the inflator 726 is disposed at the compression element 712, the hemostatic pressure in the balloons 714,716 can be adjusted after each of the sections 782a-782e have been inflated and sealed via a respective one of the clamps 786a-786e.

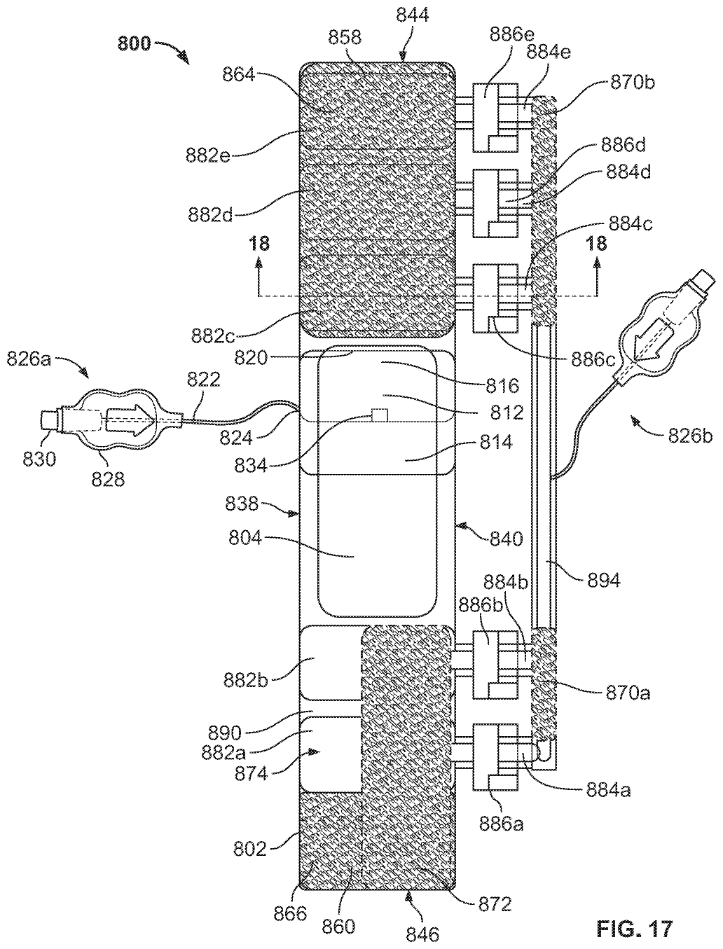

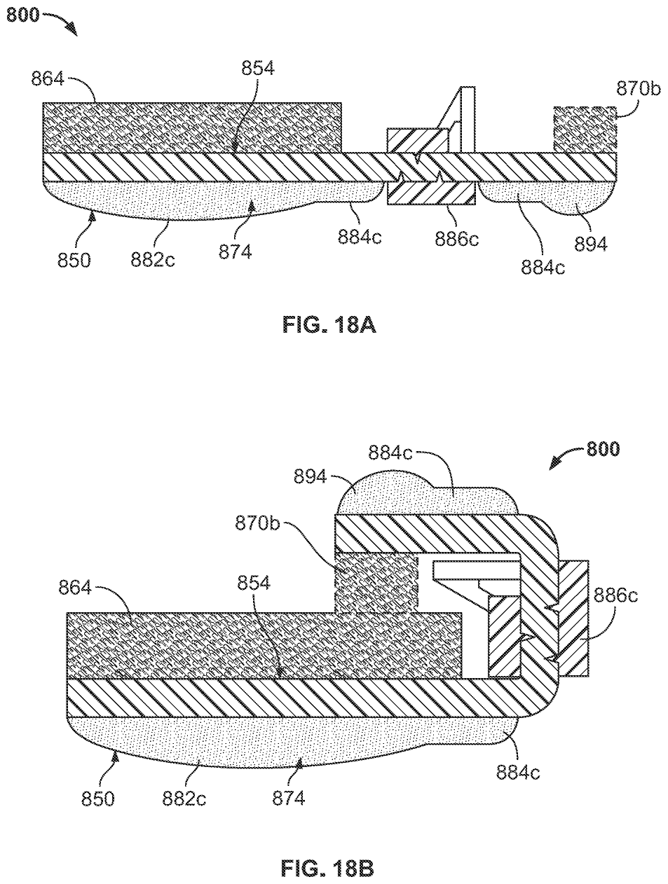

FIGS. 17, 18A, and 18B illustrate a hemostasis band 800 according to yet another embodiment of the present disclosure. In this embodiment, a main body 802 of the hemostasis band 800 is similar in design and functionality to the hemostasis band 100 shown in FIGS. 4-6, with identical or substantially-equivalent elements using reference numerals increased by a value of 700 with respect to the embodiment of FIGS. 4-6, except as described herein or as would be apparent from the figures to a person having ordinary skill in the art. Accordingly, some or all of the identical or substantially-equivalent elements of the embodiment of FIGS. 17, 18A, and 18B may not be discussed below, for sake of conciseness.

In this embodiment, main body 802 comprises a plurality of sections 882a-882e, each of which extends across substantially the entirety of the width of the main body 802 (i.e., between the first long edge 838 and the second long edge 840; see section 882c in FIGS. 18A and 18B). In this embodiment, the hemostasis band 800 further comprises a manifold 894 that is attached to a respective side edge of each of the sections 882a-882e (i.e., along second long edge 840) in fluid flow communication via respective individual channels 884a-884e. Thus, the manifold 894 connects to each of the sections 882a-882e in a “parallel” configuration such that each of the sections 882a-882e inflates substantially simultaneously when the inflator 826b is used. The manifold 894 is inflatable via an inflator 826b, and along with sections 882a-882e and channels 884a-884e the manifold 894 forms a contiguous inflatable chamber 874. Further, in this embodiment, the balloons 814,816 of the compression element 812 are inflatable via a separate inflator 826a, such that the balloons 814,816 are not in fluid flow communication with the contiguous inflatable chamber 874. In alternative embodiments, the balloons 814,816 of the compression element 812 could be provided in fluid flow communication with the contiguous inflatable chamber 874. Further, while in the embodiment illustrated the manifold 894 is disposed along the second long edge 840 of the main body 802, in alternative embodiments the manifold could be located along the first long edge 838, either short edge 844,844 (with necessary changes to the configurations of the channels having been made), or some combination of the above. In further alternative embodiments, more than one separate manifold could be provided, for example a first manifold (with individual inflator) for inflation of sections 882a,882b, and a second manifold (with individual inflator) for inflation of sections 882c-882e.

Turning back to the embodiment of FIGS. 17, 18A, and 18B, each of the channels 884a-884e is provided with a respective clamp 886a-886e located therealong, which can be used respectively to selectably seal off each of the sections 882a-882e once a desired internal pressure has been reached. In addition, once each of the sections 882a-882e and the balloons 814,816 have been inflated to a desired extent, the manifold 894 may be optionally folded over so that it is located on top of the main body 802. The manifold 894 may be optionally provided with fastener halves 870a,870b thereon that will adhere, respectively, to fastener halves 864,866 located on the main body 802, so that the manifold 894 is releasably securable in place on top of the main body 802 and not located in the clinician's way during the hemostasis procedure. In alternative embodiments, the manifold 894 may be secured in place on top of the main body 802 via tape or some other means of fixation. FIG. 18A shows a sectional view taken along line 18-18 of FIG. 17, with the manifold 894 located in an unfolded configuration as shown in FIG. 17, and FIG. 18B shows a sectional view taken alone line 18-18 of FIG. 17 with the manifold 894 folded on top of the main body 802 and secured in place using the fastener halves (fastener halves 870b,864 are shown in FIGS. 18A and 18B). Each of the clamps 886a-886e (only clamp 886c shown in FIGS. 18A and 18B) divides a respective one of the channels 882a-882e (only channel 882c shown in FIGS. 18A and 18B), with each channel 886a-886e being foldable as shown in FIGS. 18A and 18B.

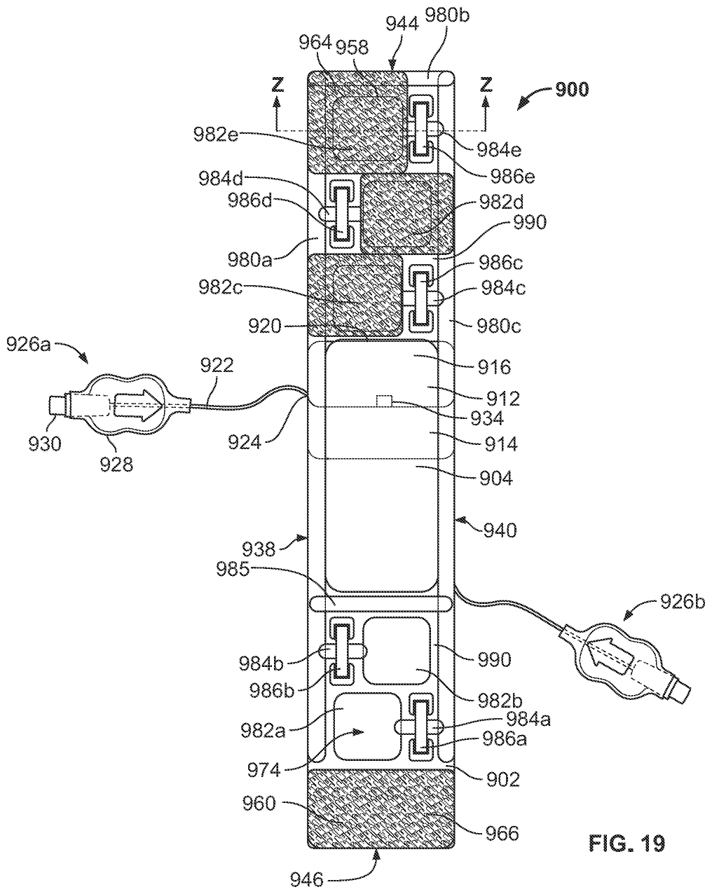

FIG. 19 illustrates a hemostasis band 900 according to yet another embodiment of the present disclosure. In this embodiment, a main body 902 of the hemostasis band 900 is similar in design and functionality to the hemostasis bands 100,500 shown in FIGS. 4-6 and 11-13, respectively, with identical or substantially-equivalent elements using reference numerals increased by a value of 800 with respect to the embodiment of FIGS. 4-6 and by a value of 400 with respect to the embodiment of FIGS. 11-13, except as described herein or as would be apparent from the figures to a person having ordinary skill in the art. Accordingly, some or all of the identical or substantially-equivalent elements of the embodiment of FIG. 19 may not be discussed below, for sake of conciseness.

The hemostasis band 900 according to the present embodiment includes a main body 902, a rigid plate 904, and a compression element 912 comprising balloons 914,916 which are inflatable via a first inflator 926a. The rigid plate 904 and the compression element 912 are substantially identical in design and function to the rigid plate 104 and the compression element 112 described above with respect to the hemostasis band 100 of FIGS. 4-6. Further, the main body 902 is substantially similar to the main body 502 of the hemostasis band 500 of the embodiment of FIGS. 11-13. For example, a cross-sectional view of the hemostasis band 900 according to the present embodiment taken through line Z-Z of FIG. 19 is substantially identical to a cross-sectional view of the hemostasis band 500 according to the embodiment of FIGS. 11-13, as seen in the cross-sectional view of FIG. 12. However, the main body 902 differs in some respects.

For example, in the embodiment of FIG. 19, the main body 902 includes a contiguous inflatable chamber 974 that comprises inflatable edge portions 980a-980c (which in this embodiment represent a continuous portion connected in fluid flow communication along the long edges 938,940 and the first short edge 944, but in alternative embodiments need not), sections 982a-982e, and channels 984a-984e. In this embodiment, each of the sections 982b,982d is directly connected to edge portion 980a along the first long edge 938 via a respective channel 984b,984d, and each of the sections 982a,982c,982e is directly connected to edge portion 980c along the second long edge 940 via a respective channel 984a,984c,984e, thus forming an alternating left-right pattern of connections along portions of the length of the main body 902 (i.e., except for the portion of the length of the main body 902 where the rigid plate 904 and balloons 914,916 are located). In alternative embodiments according to the present disclosure, the channel/section connections to the inflatable edge portions could be in any possible order, and/or the section 982e could be additionally or alternatively connected to the inflatable edge portion 980b. In this embodiment, the contiguous inflatable chamber 974 is inflated by its own individual inflator 926b, and is not in fluid flow communication with the balloons 914,916 of the compression element 912. In alternative embodiments, the balloons 914,916 of the compression element 912 could be provided in fluid flow communication with the contiguous inflatable chamber 974. Turning back to the hemostasis band 900 of FIG. 19, the contiguous inflatable chamber 974 also includes a channel 985 that connects the inflatable edge portions 980a,980c together in fluid flow communication on a side of the rigid plate 904 opposite from the section 982c, which assists with the distribution of air throughout the contiguous inflatable chamber 974. In the present embodiment, the inflatable edge portions 980a,980c effectively serve two roles, providing edge comfort and acting as a manifold system for the inflator 926b to assist with distribution of air to the contiguous inflatable chamber 974.

The hemostasis band 900 according to the present embodiment also includes a plurality of clamps 986a-986e, each of which allows for a respective one of the sections 982a-982e to be selectively separated from being in fluid flow communication with the remainder of the contiguous inflatable chamber 974 via sealing of a respective one of the channels 984a-984e based on compression thereof. Because the channels 984a-984e are arranged in parallel, each of the sections 982a-982e can be selectively inflated or sealed via opening or closing of its respective clamp 986a-986e, thus allowing for each section 982a-982e to be sealed when a desired internal pressure has been achieved.

While the principles of the claimed invention have been described above in connection with specific embodiment(s), it is to be clearly understood that this description is made only by way of example and not as a limitation of the scope of the invention, as set forth in the appended claims.

1. A hemostasis device comprising:
a main body comprising a band, the band being adapted to be wrapped and releasably secured around a body part of a patient that includes a site where bleeding is to be stopped, the band comprising an interior side that faces a skin surface of the patient when releasably secured to the body part of the patient and at least one edge portion that comprises at least a portion of a perimeter of the band, the interior side of the band comprising at least one compartment located along the at least one edge portion, the at least one compartment being inflatable; and
a compression element, the compression element comprising at least one balloon, the at least one balloon being inflatable and adapted to be placed atop the site on the body part of the patient;
wherein the band acts to direct an applied force generated by the at least one balloon when it is inflated towards the site on the body part of the patient.

2. The hemostasis device of claim 1, wherein the compression element comprises at least two inflatable balloons that press into each other when inflated to assist in the application of the applied force towards the site on the body part of the patient.

3. The hemostasis device of claim 2, wherein the at least two inflatable balloons are of different sizes.

4. The hemostasis device of claim 1, wherein the at least one edge portion of the band comprises a first long edge and a second long edge that oppose each other and form portions of the perimeter of the band, wherein each of the first long edge and the second long edge includes an inflatable compartment located along a portion of a respective length thereof.

5. The hemostasis device of claim 4, wherein each of the first long edge and the second long edge include an inflatable compartment located along a majority of a respective length thereof.

6. The hemostasis device of claim 1, wherein when the at least one compartment is inflated, an orientation of the interior side of the band with respect to the skin surface of the patient is adjusted.

7. The hemostasis device of claim 1, wherein at least portions of the band and the at least one balloon that are adapted to be placed in the vicinity of the site on the body part of the patient are transparent.

8. The hemostasis device of claim 1, the main body further comprising a plate, the at least one balloon being located at least partially between the plate and the skin surface of the patient when the least one balloon is placed atop the site on the body part of the patient, the plate being made of a material that is more rigid than the band.

9. The hemostasis device of claim 8, the plate being located at a first end of the band and comprising a means for connecting to a second end of the band, the first end and second ends being spaced apart along a length of the band.

10. The hemostasis device of claim 1, further comprising at least one inflatable section that is not located along a portion of the perimeter of the band.

11. The hemostasis device of claim 10, wherein the at least one inflatable section is connected in fluid flow communication with the at least one edge portion via a channel.

12. The hemostasis device of claim 11, further comprising a clamp, valve, or stopcock located along the channel, the clamp, valve, or stopcock permitting the at least one inflatable section to be opened to or closed from fluid flow communication with the at least one edge portion.

13. The hemostasis device of claim 1, wherein the at least one edge portion is in fluid flow communication with the compression element.

14. A hemostasis device comprising:
a band, the band being adapted to be wrapped and releasably secured around a body part of a patient that includes a site where bleeding is to be stopped, the band comprising an interior side that faces a skin surface of the patient when releasably secured to the body part of the patient, the interior side of the band comprising a contiguous inflatable chamber, the contiguous inflatable chamber comprising at least one edge portion located along at least a portion of a perimeter of the band; and
a compression element comprising at least one balloon, the at least one balloon being inflatable and adapted to be placed atop the site on the body part of the patient;
wherein the at least one band acts to direct an applied force generated by the at least one balloon when it is inflated towards the site on the body part of the patient.

15. The hemostasis device of claim 14, wherein the contiguous inflatable chamber further comprises at least one inflatable section that is not located along a portion of the perimeter of the band.

16. The hemostasis device of claim 15, wherein the at least one inflatable section is connected in fluid flow communication with the at least one edge portion via a channel.

17. The hemostasis device of claim 16, further comprising a clamp, valve, or stopcock located along the channel, the clamp, valve, or stopcock permitting the at least one inflatable section to be opened to or closed from fluid flow communication with a remainder of the contiguous inflatable chamber.

18. The hemostasis device of claim 14, wherein the contiguous inflatable chamber is in fluid flow communication with the compression element.

19. The hemostasis device of claim 14, wherein the contiguous inflatable chamber cannot be placed in fluid flow communication with the compression element, the hemostasis device further comprising a first inflator attached in fluid flow communication with the compression element and being adapted to allow for inflation of the at least one balloon, and a second inflator attached in fluid flow communication with the contiguous inflatable chamber and being adapted to allow for inflation of the contiguous inflatable chamber.

20. The hemostasis device of claim 14, wherein when the contiguous inflatable chamber is inflated, an orientation of the interior side of the band with respect to the skin surface of the patient is adjusted.
